(19) United States
(12) Patent Application Publication
(10) Pub. No.: US 2021/0285936 A1
(43) Pub. Date: Sep. 16, 2021

(54) DOCKED APTAMER EAB BIOSENSORS

(71) Applicant: Eccrine Systems, Inc., Cincinnati, OH (US)

(72) Inventors: Robert Beech, Montgomery, OH (US); Mikel Larson, Cincinnati, OH (US); Jacob A Bertrand, Montgomery, OH (US); Gavi Begtrup, Cincinnati, OH (US)

(73) Assignee: Eccrine Systems, Inc., Cincinnati, OH (US)

(21) Appl. No.: 16/624,062

(22) PCT Filed: Jun. 25, 2018
(86) PCT No.: PCT/US18/39274

§ 371 Date: Dec. 18, 2019

Related U.S. Application Data
(60) Provisional application No. 62/523,835, filed on Jun. 23, 2017.

(51) Int. Cl. G01N 33/53; G01N 33/543; G01N 27/327

(52) U.S. Cl. CPC G01N 33/5308; G01N 33/5438; G01N 2458/30; G01N 27/3277; G01N 33/54386

(57) ABSTRACT

Electrochemical aptamer-based biosensing devices and methods are described herein that are configured to produce a detectible signal upon target analyte interaction, with reduced reliance on a conformational change by the aptamer. The disclosure includes embodiments of docked aptamer EAB sensors for measuring the presence of a target analyte in a biofluid sample. The sensors include an electrode capable of sensing redox events, and a plurality of aptamer sensing elements with aptamers selected to interact with a target analyte. Each aptamer sensing element includes a molecular docking structure attached to the electrode, and an analyte capture complex that includes an aptamer releasably bound to the docking structure, and an electroactive redox moiety. Upon the aptamer binding with a target analyte, the analyte capture complex separates from the docking structure. The separation of the analyte capture complex from the docking structure produces a positional change in the redox moiety that is detectable by the sensing device on interrogation of the electrode.

### CROSS-REFERENCE TO RELATED APPLICATIONS

The present application claims priority to PCT/US18/39274, filed Jun. 25, 2018, and U.S. Provisional Application Ser. No. 62/523,835, filed Jun. 23, 2017, and has specification that builds upon PCT/US17/23399, filed Mar. 21, 2017, the disclosures of which are hereby incorporated herein by reference in their entirety.

### BACKGROUND OF THE INVENTION

Despite the many ergonomic advantages of perspiration (sweat) compared to other biofluids (particularly in “wearable” devices), sweat remains an underutilized source of biomarker analytes compared to the established biofluids: blood, urine, and saliva. Upon closer comparison to other non-invasive biofluids, the advantages may even extend beyond ergonomics: sweat might provide superior analyte information. Several challenges, however, have kept sweat from occupying its place among the preferred clinical biofluids. These challenges include very low sample volumes (nL to μL), unknown concentration due to evaporation, filtration and dilution of large analytes, mixing of old and new sweat, and the potential for contamination from the skin surface. More recently, rapid progress in “wearable” sweat sampling and sensing devices has resolved several of the historical challenges. However, this recent progress has also been limited to high concentration analytes (μM to mM) sampled at high sweat rates (>1 nL/min/gland) found in, for example athletic applications. Progress will be much more challenging as biosensing moves towards detection of small proteins, and large, low concentration analytes (nM to pM and lower).

In particular, many known sensor technologies for detecting small molecules are ill-suited for use in wearable biofluid sensing, which requires sensors that permit continuous use on a wearer's skin. This means that sensor modalities that require complex microfluidic manipulation, the addition of reagents, the use of limited shelf-life components, such as antibodies, or sensors that are designed for a single use will not be sufficient for many biofluid sensing applications. Electrochemical aptamer-based (“EAB”) sensor technology, such as is disclosed in U.S. Pat. Nos. 7,803,542 and 8,003,374, presents a stable, reliable bioelectric sensor that is sensitive to the target analyte in biofluid, while being capable of multiple analyte capture events during the sensor lifespan. However, a chief obstacle to the development of such sensors is the ability to select suitable aptamers capable of capturing, and by extension allowing the sensor to detect, target analytes.

The state of the art technology for aptamer selection relies on techniques, such as systematic evolution of ligands by exponential enrichment (“SELEX”) processes, that iteratively select for aptamers having the desired capture properties for the target analyte. One such SELEX process works by tethering target molecules to a substrate, and then washing the tethered analytes with a library of about 10₁₄ different aptamer sequences. The non-binding aptamers are removed, and the aptamers that successfully bonded to the target analyte are polymerase chain reaction (“PCR”) amplified and reintroduced to the target analyte. After several iterations, candidate aptamers that preferentially bind to the target analyte will emerge.

These candidate aptamers are then functionalized into prior art multi-capture aptamer sensing elements, as depicted in FIG. 1A, which will detect the presence of the target analyte in a biofluid by interacting with analytes as they contact the aptamer, and then releasing the analyte back into the biofluid after an interval. The sensing element 110 includes an analyte capture complex 112 that includes a randomized aptamer 140 selected to interact with a target analyte, and may have one or more linker nucleotide sections 142. The analyte capture complex has a first end covalently bonded to a dock 120, e.g., a sulphur-based molecule such as a thiol, which is in turn covalently bonded to an electrode base 130. The sensing element further includes a redox moiety 150 that may be covalently bonded to the analyte capture complex 112 or bound to it by a linking section. In the absence of the target analyte, the aptamer 140 is in a first configuration, and the redox moiety 150 is in a first position relative to the electrode 130. When the device interrogates the sensing element using, for example, square wave voltammetry (SWV), the sensing element produces a first electrical signal, eTA.

With reference to FIG. 1B, when the aptamer 140 interacts with a target analyte 160, the aptamer undergoes a conformation change that partially disrupts the first configuration, and forms a second configuration. The interaction with the target analyte 160 accordingly moves the redox moiety 150 into a second position relative to the electrode 130. Now when the biofluid sensing device interrogates the sensing element, the sensing element produces a second electrical signal, eT_{B}, that is distinguishable from the first electrical signal, eT_{A}. After a recovery interval, the aptamer releases the target analyte back into the biofluid solution, and the aptamer will return to the first configuration, which will produce the corresponding first electrical signal when the sensing element is interrogated. The sensing element is now capable of interacting with another target analyte.

Unfortunately, most SELEX processes only identify candidate aptamers that preferentially bind to the target analyte. They do not select for aptamers that display a conformational change sufficient to produce an analyte capture signal that is distinguishable from the unbound signal. Such selection instead is done though an intensive trial and error process that involves functionalizing the candidate aptamers and testing their performance empirically. Not only is this process time-consuming, but in the end, it may not produce a suitable aptamer. Clearly, aptamer selection and EAB sensor configuration requires improvement if EAB sensing is to become practical for wearable biofluid sensing. Accordingly, there is a need for new EAB sensor configurations and methods of detecting analyte capture with selected aptamers. In particular, there is a need for sensing devices having selected aptamers that not only preferentially bind to a target analyte, but also produce a reliably detectable signal upon analyte capture. Such devices and methods are the subject of the present disclosure.

Many of the other challenges to successful biofluid sensor development can be resolved by creating novel and advanced interplays of chemicals, materials, sensors, electronics, microfluidics, algorithms, computing, software, systems, and other features or designs, in a manner that affordably, effectively, conveniently, intelligently, or reliably brings biofluid to sensors and sample preparing or concentrating subsystems.

### SUMMARY OF THE INVENTION

Electrochemical aptamer-based biosensing devices and methods are described herein that are configured to produce a detectible signal upon target analyte interaction with reduced reliance on a conformational change by the aptamer. In the disclosed devices, aptamers can be selected for preferential binding with a target analyte, with reduced reliance on the further step of selecting for a detectable conformation change in the presence of the analyte. Embodiments are disclosed herein for an EAB sensor with docked aptamers for measuring the presence of a target analyte in a biofluid sample. In the disclosed embodiments, the sensor includes an electrode capable of sensing redox events, and a plurality of aptamer sensing elements with aptamers selected to interact with a target analyte. Each aptamer sensing element includes a molecular docking structure attached to the electrode, and an analyte capture complex that includes an aptamer releasably bound to the docking structure, and an electroactive redox moiety. Upon the aptamer binding with a target analyte, the analyte capture complex separates from the docking structure. The separation of the analyte capture complex from the docking structure produces a positional change in the redox moiety that is detectable by the sensing device on interrogation of the electrode.

### BRIEF DESCRIPTION OF THE DRAWINGS

The present disclosure will be further appreciated in light of the following detailed descriptions and drawings in which:

FIGS. 1A and 1B are representations of a previously-disclosed aptamer sensing element;

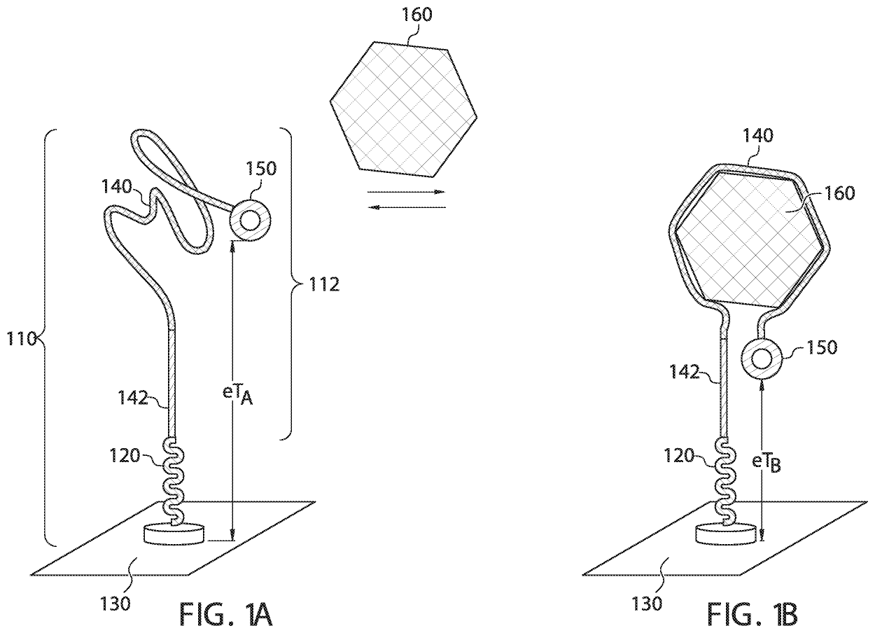

FIGS. 2A and 2B depict an exemplary embodiment of a docked aptamer sensing element before and after analyte capture;

FIGS. 3A and 3B depict an exemplary embodiment of a docked aptamer sensing element before and after analyte capture;

FIGS. 4A and 4B depict an exemplary embodiment of a docked aptamer sensing element before and after analyte capture;

FIG. 5 depicts an exemplary embodiment of a docked aptamer EAB sensor, in which multiple aptamer sensing elements are depicted before and after analyte capture; and

FIG. 6 depicts an exemplary embodiment of a docked aptamer EAB sensor, in which multiple aptamer sensing elements are depicted before and after analyte capture.

### DEFINITIONS

Before continuing with a detailed description of the exemplary embodiments, a variety of definitions should be made, these definitions gaining further appreciation and scope in the detailed description and embodiments of the present disclosure.

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva.

“Biofluid sensor” means any type of sensor that measures a state, presence, flow rate, solute concentration, solute presence, in absolute, relative, trending, or other ways in a biofluid. Biofluid sensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a biofluid sensing device.

“Measured” can imply an exact or precise quantitative measurement and can include broader meanings such as, for example, measuring a relative amount of change of something. Measured can also imply a binary or qualitative measurement, such as ‘yes’ or ‘no’ type measurements.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

“EAB sensor” means an electrochemical aptamer-based biosensor that is configured with multiple aptamer sensing elements that, in the presence of a target analyte in a biofluid sample, produce a signal indicating analyte capture, and which signal can be added to the signals of other such sensing elements, so that a signal threshold may be reached that indicates the presence of the target analyte.

“Analyte capture complex” means an aptamer, or other suitable molecules or complexes, such as proteins, polymers, molecularly imprinted polymers, polypeptides, and glycans, that experience a conformation change in the presence of a target analyte, and are capable of being used in an EAB sensor. Such molecules or complexes can be modified by the addition of one or more linker sections comprised of nucleotide bases.

“Aptamer sensing element” means an analyte capture complex that is functionalized to operate in conjunction with an electrode to detect the presence of a target analyte. Such fractionalization may include tagging the aptamer with a redox moiety, or attaching thiol binding molecules, docking structures, or other components to the aptamer or capture complex. Multiple aptamer sensing elements functionalized on an electrode comprise an EAB sensor.

“Multi-capture Aptamer Sensor” means an EAB sensor capable of a plurality of analyte capture interactions, as disclosed in U.S. Pat. Nos. 7,803,542 and 8,003,374, which are hereby incorporated herein in their entirety.

“Docked aptamer EAB sensor” means an EAB sensor that employs docking strategies to connect analyte capture complexes with the sensor electrode, and wherein such analyte capture complexes are configured for one analyte capture interaction.

“Reference EAB sensor” means a reference sensor that comprises aptamer sensing elements functionalized on an electrode base, where the aptamers have been modified to not interact with target analyte molecules. A reference EAB sensor is configured to perform substantially identically to a comparable active EAB biosensor but will not bind to a target analyte.

“Sensitivity” means the change in output of the sensor per unit change in the parameter being measured. The change may be constant over the range of the sensor (linear), or it may vary (nonlinear).

“Signal threshold” means the combined strength of signal-on indications produced by a plurality of aptamer sensing elements that indicates the presence of a target analyte.

“Time-to-threshold” means the amount of time required for an EAB sensor to reach signal threshold. Such time may be calculated from the initiation of device use, the initiation of sweating, a sensor regeneration time, or other suitable starting point.

### DETAILED DESCRIPTION OF THE INVENTION

The embodiments described herein solve a shortcoming of SELEX processes with respect to biofluid sensor development through the use of docked aptamer EAB sensors. The disclosed sensors reduce the requirement of identifying aptamers that not only preferentially bind to the target analyte, but also display a conformational change sufficient to produce an analyte capture signal that is distinguishable from the unbound signal. Rather, the docked aptamer sensors use a change in position of a redox moiety, caused by detachment of a bound aptamer from a docking structure, to detect analyte capture. As described herein, a docked aptamer EAB sensor includes an aptamer initially bound to a docking structure. While bound to the docking structure, a redox moiety associated with the aptamer produces a first redox signal measurable by the sensing device. Upon interaction with the analyte, the aptamer changes shape to bind with the analyte, causing the aptamer to break away from the docking structure. The separation of the aptamer from the docking structure produces a change in the location of the associated redox moiety. The change in redox moiety location produces a second redox signal that is measurably different from the first redox signal. The difference between the first and second measured redox signals can be correlated and compared to a threshold to detect analyte capture.

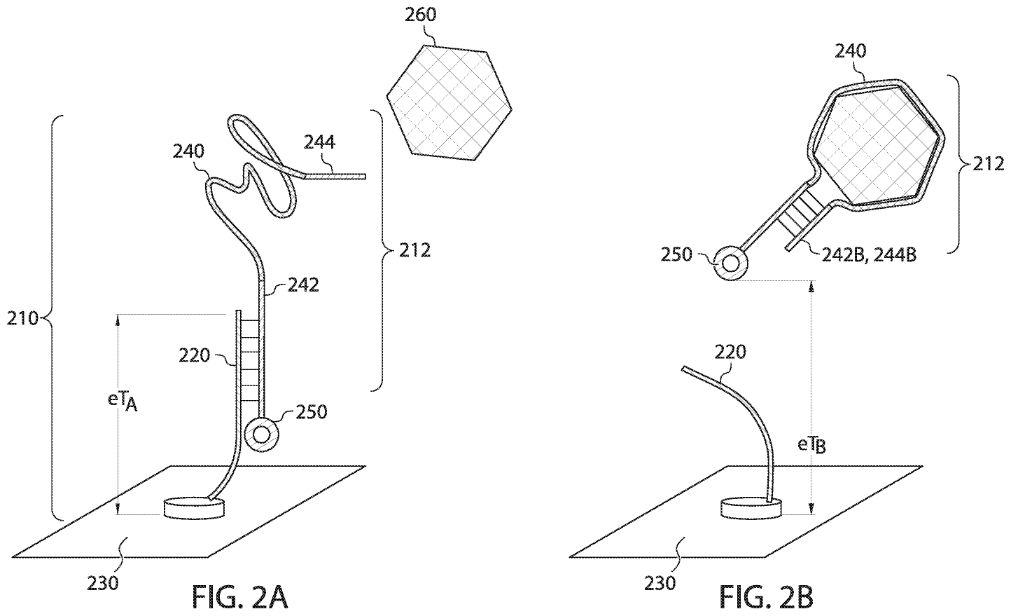

Turning now to FIGS. 2A and 2B, which depict a first embodiment of a docked aptamer sensing element. In this embodiment, an aptamer sensing element 210 includes an analyte capture complex 212 and a molecular docking structure 220 immobilized on an electrode 230. While the figures depict, and the discussion focuses on, a single aptamer sensing element, EAB sensors in each of the exemplary embodiments described herein will include a large number of such aptamer sensing elements (thousands, millions, or billions of individual sensing elements, having an upper limit of 1014/cm2) attached to the electrode. In turn, the disclosed EAB sensor is configured to be used within a biofluid sensing device. The docking structure 220 may be attached to the electrode 230 by covalently bonding a first end to a thiol, which is then covalently bonded to the electrode. The electrode 230 may be comprised of gold, copper, carbon, functionalized polymer, biotinylated beads, other beads, or another suitable conductive material. The docking structure 220 can include a 9 to 12 base nucleotide sequence that is selected to be complementary with a nucleotide sequence on the analyte capture complex 212, specifically, the dock is configured to pair with a first linker section 242.

The aptamer sensing element 210 also includes an analyte capture complex 212 for binding to a target analyte 260. The analyte capture complex includes an aptamer 240 selected to bind to a target analyte, and may also include one or more linker nucleotide sections, here depicted as a complementary pair of linkers 242, 244. The complementary linkers 242, 244 may be of differing lengths, specifically, the first linker 242 may include more nucleotide bases than the second linker 244. Such an arrangement makes the second linker competitive to the binding between the dock 220 and first linker. In this embodiment, a redox chemical moiety 250 such as, for example, a methylene blue group, a viologen, or a ferrocene group, is attached to the free end of the first linker 242.

In the initial arrangement, shown in FIG. 2A, when the EAB sensor is assembled, the analyte capture complex 212 with attached redox moiety 250 are placed in a solution that is washed over a dock 220 that is attached to the electrode 230. In the absence of target analytes, the first linker 242 requires less energy to bind to the docking sequence than it requires to bind to the second linker 244, and accordingly attaches the analyte capture complex to the dock to form an aptamer sensing element. When attached to the dock, the analyte capture complex is configured so that the redox moiety 250 is located in close proximity to the electrode 230. The distance between the redox moiety and electrode is sufficiently small for facile electron transduction (eTA), thereby enabling redox of the redox moiety in response to potentials applied via the electrode. In operation, the EAB sensor is exposed to a biofluid sample containing a concentration of the target analyte 260.

With reference to FIG. 2B, on interaction with the target analyte 260, the aptamer 240 physically changes its three dimensional structure to bind with the analyte. As the aptamer interacts with the analyte, the second linker 244B moves into physical proximity to the first linker 242B. The physical proximity of the complementary linkers causes the first linker to break free from the docking sequence 220 and to bind with the second linker. As the analyte capture complex 212 breaks away from the docking structure 220, the complex diffuses into the bulk of the biofluid and then is carried away from the electrode 230 by the biofluid flow, causing the attached redox moiety 250 to also move away from the electrode. As the redox moiety moves away from electrode, the increased distance causes the electron transfer (eT_{B}) between the redox moiety and the electrode to decrease significantly. Accordingly, interrogation of the electrode 230 will return no signal or reduced signal due to the absent or reduced electron transfer with the redox moiety 250. In this embodiment, the EAB sensor is a signal-off sensor, since capture of a target analyte 260 causes a signal reduction to be reported from the sensor to the biofluid sensing device.

Because this embodiment is a signal-off sensor, it can be vulnerable to false positives caused by physical degradation of the individual aptamer sensing elements, or changes in conditions. Over time, aptamer sensing elements within an EAB sensor will physically degrade, meaning docks will release from the electrode and the sensing elements will become unattached to the electrode surface independent of target analyte concentration in the biofluid. Further, docked aptamer sensors have a second source of degradation since the analyte capture complexes will gradually detach from their respective docks independent of target analyte concentration. Similarly, changes in external or internal temperature, humidity, non-specific binding factors, and biofluid sample pH and salinity can affect the rate at which the sensors degrade. Therefore, some embodiments of the disclosed invention further include a reference EAB sensor to provide drift correction and calibration for a companion active EAB sensor. While reference EAB sensors are discussed here in the context of signal-off and docked aptamer EAB sensors, their use is not so limited, and other types of active EAB sensors, including multi-capture EAB sensors, can benefit from appropriately configured companion reference sensors.

An embodiment of the disclosed reference EAB sensor is configured to be substantially identical to its companion active EAB sensor, however, the aptamer is altered so that it will no longer interact with the target molecule. By adhering as closely as possible to the companion active sensor's physical characteristics, the reference sensor will mirror the drift or physical degradation experienced by the active sensor due to time or conditions. Like its companion sensor, the reference sensor will include an electrode, and a plurality of aptamer sensing elements, each including a deactivated aptamer, and a redox moiety. A deactivated aptamer may be produced by, e.g., switching out one of more nucleotide bases in the aptamer to render it incapable of interacting with the target analyte without substantially altering the aptamer's structural configuration. In use, the reference EAB sensor will allow the biofluid sensing device to chart the drift or physical degradation of its companion active sensor. The reference EAB sensor can also perform an initial diagnostic test of a device by providing a measurement of physical degradation within the active sensor that has occurred up until the time of use.

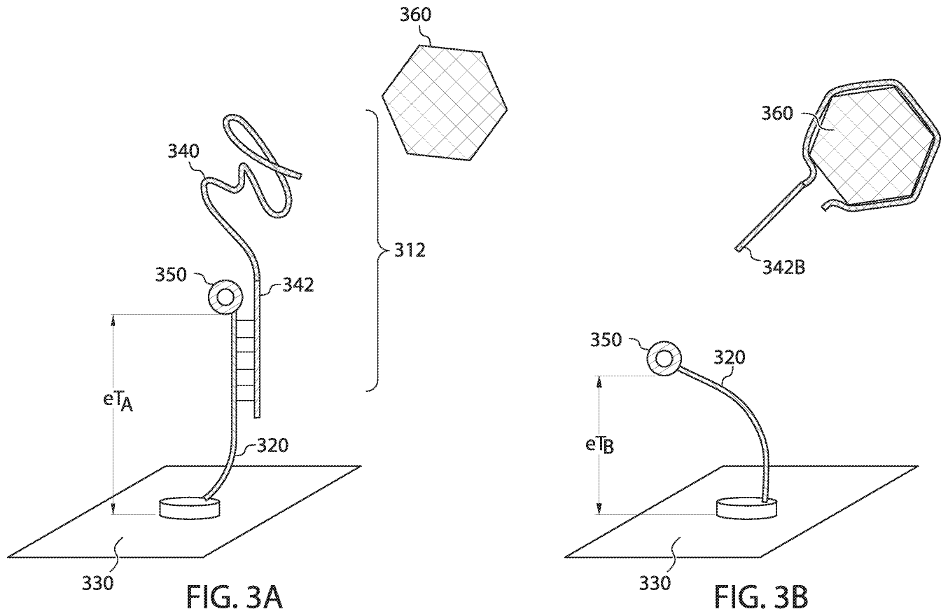

FIGS. 3A and 3B depict another embodiment of an active docked aptamer sensing element. This embodiment is similarly configured to the embodiment depicted in FIG. 2, however, this variation includes a redox chemical moiety 350 that is immobilized on the unattached end of the dock 320, on the opposite end of the dock from the electrode 330. This embodiment also includes a single linker sequence 342, as may be used in certain embodiments described herein. In the initial arrangement, shown in FIG. 3A, during EAB sensor assembly, the analyte capture complex 312 binds to the dock 320, which is thereby stiffened so that the redox moiety 350 is located at a distance from the electrode 330, being approximately the full length of the dock. The distance between the redox moiety and electrode is sufficiently large to prevent most electron transduction, thereby largely preventing redox of the redox moiety in response to potentials applied via electrode 330, effectively creating a no- or reduced-signal baseline condition prior to analyte capture eT_{A}.

In operation, the EAB sensor is exposed to a biofluid sample containing a concentration of the target analyte 360. On interaction with the target analyte, the aptamer changes shape to bind with the analyte, causing the linker 342 to break free from the dock 320, and the complex moves away from the dock, as shown in FIG. 3B.

Once the dock 320 is free from the linker 342B, the dock becomes more flexible, and begins to move freely about its point of attachment to the electrode. As the attached redox moiety 350 moves about the dock attachment point, the redox moiety moves sufficiently close to the electrode to promote a detectable electron transduction, eT_{B}. Interrogation of the electrode 330 following analyte capture, therefore, will return a detectable signal due to movement of the redox moiety 350 closer to the electrode. In this embodiment, the EAB sensor has a signal-off condition prior to analyte capture and a signal-on condition after analyte capture, enabling a positive detected signal to provide confirmation of analyte capture.

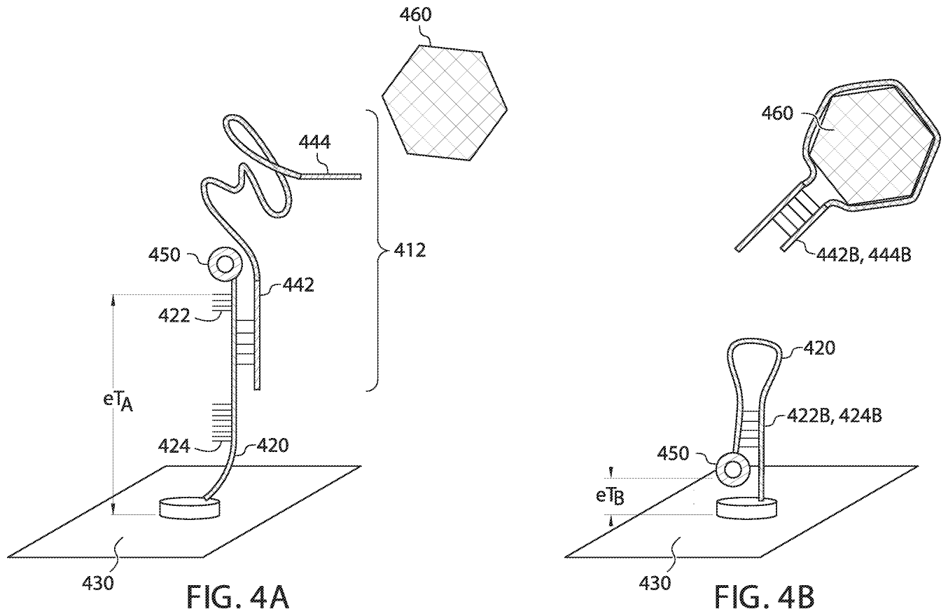

FIGS. 4A and 4B depict a third embodiment of a docked aptamer sensing element. This embodiment has a dock that is configured similarly to the embodiment depicted in FIG. 3, having a redox chemical moiety 450 immobilized on the unattached end of the dock 420, on the opposite end of the dock from the electrode 430. In addition, in this embodiment, the dock further includes two complementary nucleotide sequences 422, 424. During sensor assembly, when the docks are attached or annealed to the electrode, some of the complementary sections will bind to each other prematurely. Therefore, one or more purification steps may be required to remove such bound docks and attach additional unbound docks prior to attaching the analyte capture complexes. As shown in FIG. 4A, after the dock is annealed to the electrode, the analyte capture complex 412 binds to the dock 420, which is thereby stiffened so that the redox moiety 450 is located at a distance from the electrode 430, being approximately the full length of the dock. The distance between the redox moiety and electrode is sufficiently large to prevent most electron transduction, thereby largely preventing redox of the redox moiety in response to potentials applied via electrode 430, effectively creating a no- or reduced-baseline signal condition prior to analyte capture eT_{A}.

In operation, the EAB sensor is exposed to a biofluid sample containing a concentration of the target analyte 460. On interaction with the target analyte, the aptamer changes shape to bind with the analyte, causing the second linker 444 to move into physical proximity to the first linker 442. The physical proximity of the complementary linkers causes the first linker to break free from the dock 420 and bind to the second linker 444, and the complex is carried away from the docking structure 420, as shown in FIG. 4B.

Once the dock 420 is unbound from the first linker 442B, the dock becomes more flexible, and the complementary sections 422B, 424B bind together. The folding of dock 420 caused by the sections binding locks the attached redox moiety 450 in a position close to the electrode 430, thereby promoting a detectable electron transduction, eT_{B}. Interrogation of the electrode 430 following analyte capture, therefore, will return a detectable signal due to the proximity of the redox moiety to the electrode. In this embodiment, the EAB sensor has a signal-off condition prior to analyte capture and a signal-on condition after analyte capture, enabling a positive detected signal to provide confirmation of analyte capture. Relative to the embodiment depicted in FIG. 3, however, the detectable signal will remain more consistent over repeated interrogations of the electrode, i.e., less noisy, due to the fixed position of the redox moiety.

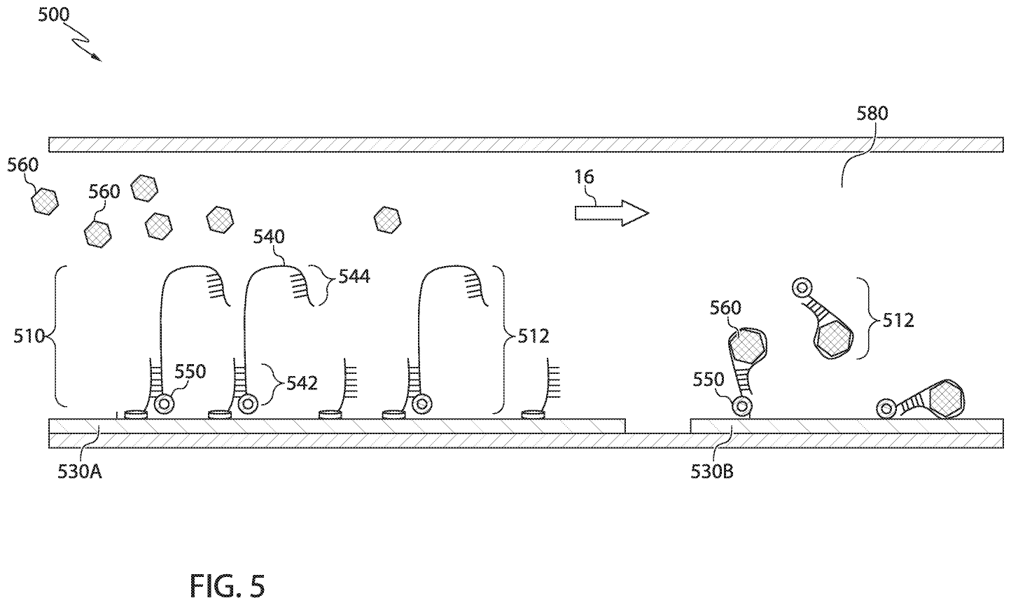

FIG. 5 depicts an alternate embodiment of a docked aptamer EAB sensor 500 featuring an alternate signal detection configuration. In this embodiment, the sensor includes a first electrode 530A, and a second electrode 530B, both located within a microfluidic channel 580 for collecting and conveying one or more biofluid samples, e.g., eccrine sweat, as the sample emerges from the skin. The channel has a first end that is upstream relative to the flow of biofluid, shown by the arrow 16, and a second end, which is downstream relative to the biofluid flow direction. The second electrode 530B is therefore located downstream of the first electrode relative to a biofluid flow direction 16.

In an initial arrangement, the first electrode 530A is configured with a multitude of aptamer sensing elements, each of which includes a docking structure 520 immobilized on the electrode, an analyte capture complex 512, and a redox moiety 550, here shown bonded to the free end of a first linker 542. The dock 520 may be attached to the electrode 530A by covalently bonding a first end to a thiol, which is then in turn covalently bonded to the electrode, which may be comprised of gold or another suitable conductive material. The aptamer sensing elements may be arranged in any manner described in the previous embodiments, and here they are depicted as similar to those described in FIGS. 3A and 3B. The dock 520 includes a 9 to 12 base nucleotide sequence that is selected to be complementary to the first linker section 542. The analyte capture complex 512 includes an aptamer 540 selected for binding to a target analyte 560, as well as one or more linkers, here a complementary pair 542, 544 is shown. In this configuration, the redox moiety 550 is placed in close proximity to the first electrode 530A, thereby enabling redox of the redox moiety in response to potentials applied via the first electrode 530A. In the absence of target analytes, this embodiment will therefore have a signal-on condition at the first electrode 530A, and a signal-off condition at the second electrode 530B.

In operation, the sensor 500 is exposed to a biofluid sample containing a concentration of target analytes 560. As the biofluid sample flows through the channel 580 in the direction of the arrow 16, target analyte molecules interact with the aptamers 540, causing the second linker 544 to move into physical proximity to the first linker 542. The first linker then breaks free from the dock 520, and binds to the second linker. The analyte capture complex 512 then moves away from the dock, causing the attached redox moiety 550 to also move away from the first electrode. As the redox moiety 550 moves away from the first electrode, the electron transfer between the redox moiety and the first electrode is blocked due to the distance between the two, creating a decreasing signal condition at the first electrode.

After separation, the freed analyte capture complex 512 and captured analyte 560 are carried as a unit by the biofluid through the fluid channel 580 in the sample flow direction 16. As the analyte capture complexes 512 move away from the first electrode 530A and toward the second electrode 530B, a number of the complexes will approach the second electrode 530B so that the second electrode registers a signal. As individual complexes approach the second electrode, the proximity of the redox moiety 550 to the second electrode will enable redox of the redox moiety in response to potentials applied via the second electrode. The increase from no redox signal to a measurable signal will be detected through the second electrode as an indication of analyte capture. The decreasing signal at the first electrode 530A, combined with increasing signal at the second electrode 530B will provide an indication of the concentration or presence of target analyte in the sample.

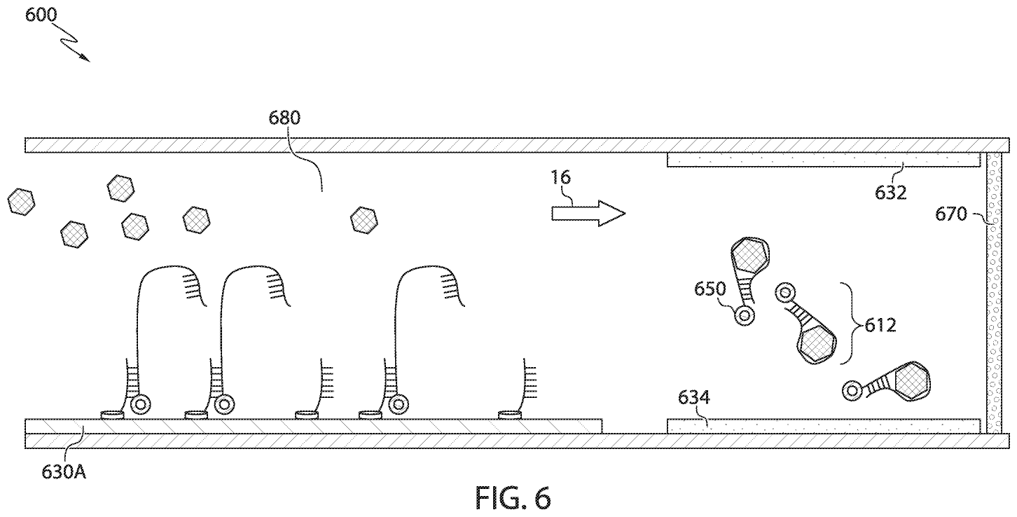

FIG. 6 depicts a variation of the embodiment described in relation to FIG. 5. In this embodiment, instead of a second electrode 530B, the device has a pair of electrodes 632, 634 located downstream of the first electrode 630A with respect to the flow direction 16. The paired electrodes 632, 634 create a voltage across the channel 680 nearest the channel's second end. The paired electrodes interrogate the channel between them and detect the presence of any redox moieties 650 that move into the area. Some versions of this embodiment, as well as the embodiment depicted in FIG. 5, may include a filter 670 at the second end of the channel 680 to increase the concentration of analyte between the paired electrodes 632, 634. The filter 670 may be any selectively permeable membrane, e.g., an osmosis membrane, a dialysis membrane, a gel, or other suitable material, so long as water is allowed to pass through and the analyte capture complexes 612 are retained between the paired electrodes. Rather than a filter, some embodiments may have a cap that substantially stops flow of biofluid through the channel, causing analyte capture complexes to concentrate between the paired electrodes.

With each of the embodiments depicted above, the interactions among the dock and the one or more linker sections may prove critical to the performance of the docked aptamer sensors. Therefore, linker and dock length and composition may be adjusted to improve or allow sensor function. Relative bond strength can be adjusted by varying the nucleotide bases (adenine (A), thymine (T), guanine (G), cytosine (C), uracil (U)) in the linkers and dock, to include adding non-native or unnatural bases. For example, an A-T bond is weaker than a G-C bond. By creating relatively more G-C complementary pairs between a linker and a dock, a stronger bond can be created. Similarly, placing a G-C pair at the end of the linker-dock complex creates a stronger bond. Conversely, the inclusion of more A-T complementary pairs produces a relatively weaker bond between dock and linker. Bond strength can also be adjusted by making the length of a component longer or shorter. For example, two complementary linkers that are 9 bases each would have a stronger bond relative to two complementary 3 base linkers.

Adjusting these parameters will allow adjustment of EAB sensitivity and drift. For example, a strong bond between a dock and its associated analyte capture complex may increase sensor lifespan (reduce drift) by reducing sensor degradation over time, i.e., by slowing analyte capture complex detachment from its dock. However, stronger bonds between docks and analyte capture complexes could also reduce sensitivity, e.g., analyte capture produces an insufficient conformation change to disrupt the bond with the dock, and no signal is produced.

In addition to the description above, sensing devices may be further configured for improved performance in low-concentration detection. For example, one or more filtering membranes can be placed before and after the electrodes, or the sensors may be electromagnetically shielded to reduce the effects of electrical noise, thereby improving sensitivity. Similarly, an EAB sensing element may be surrounded by neutral pH fluid to improve sensitivity.

While several exemplary embodiments have been described with reference to a molecular docking structure, it is anticipated that other types of docking structures may also be used, provided the docking structure is designed to release an aptamer upon binding of the aptamer to a target analyte. Various modifications, alterations, and adaptations to the embodiments described herein may occur to persons skilled in the art with attainment of at least some of the advantages. The disclosed embodiments are therefore intended to include all such modifications, alterations, and adaptations without departing from the scope of the embodiments as set forth herein.

This has been a description of the disclosed invention along with a preferred method of practicing the disclosed invention, however the invention itself should only be defined by the appended claims.

1. A sensing device, comprising:
a plurality of aptamer sensing elements for indicating the presence of a target analyte in a fluid sample, each sensing element comprising:
a dock configured to attach to the electrode, the dock comprising a dock nucleotide sequence, and an electrode bonding molecule;
an analyte capture complex, wherein a portion of the analyte capture complex is complementary to at least a portion of the dock nucleotide sequence, the analyte capture complex comprising a randomized aptamer sequence selected to interact with the target analyte, and one or more linker nucleotide sequences; and
an electroactive redox moiety; and
an electrode configured to detect redox events from the electroactive redox moiety

2. The sensing device of claim 1, wherein the electroactive redox moiety is bound to one of the following: the dock, and the analyte capture complex.

3. The sensing device of claim 1, wherein the analyte capture complex further comprises a first linker nucleotide sequence and a second linker nucleotide sequence, and wherein at least a portion of the first linker sequence is complementary to the second linker sequence.

4. The sensing device of claim 3, wherein the electroactive redox moiety is bound to one of the following: the first linker sequence, the second linker sequence, and the dock.

5. The sensing device of claim 1, wherein the dock nucleotide sequence further comprises: a first dock nucleotide sequence, a second dock nucleotide sequence, and a third dock nucleotide sequence, and wherein at least a portion of the first nucleotide sequence is complementary to at least a portion of the second nucleotide sequence, and wherein at least a portion of the third dock nucleotide sequence is complementary to a portion of the analyte capture complex.

6. The sensing device of claim 3, wherein the first linker nucleotide sequence includes more nucleotides than the second linker nucleotide sequence, and wherein at least a portion of the first linker sequence is complementary to at least a portion of the dock nucleotide sequence.

7. The sensing device of claim 1, further comprising a reference sensor, comprising:
a plurality of reference aptamer sensing elements, each reference sensing element comprising:
a dock configured to attach to the electrode, the dock comprising a dock nucleotide sequence, and an electrode bonding molecule;
a reference analyte capture complex, wherein a portion of the reference analyte capture complex is complementary to at least a portion of the dock nucleotide sequence, the analyte capture complex comprising a deactivated randomized aptamer sequence, and one or more linker nucleotide sequences; and
an electroactive redox moiety; and
an electrode configured to detect redox events from the electroactive redox moiety

8. The device of claim 1, wherein the fluid sample is one of the following: sweat, interstitial fluid, blood, plasma, serum, tears, and saliva.

9. A sensing device configured to receive a fluid sample, the sensing device comprising:
a channel having a first channel end, and a second channel end, and wherein the fluid sample moves through the channel from the first channel end to the second channel end;
a first electrode and a second electrode, wherein the first electrode and second electrode are configured to detect redox events, and wherein the first electrode is located closer to the first channel end than to the second channel end, and the second electrode is located closer to the second channel end than to the first channel end;
a plurality of aptamer sensing elements for indicating the presence of a target analyte in the fluid sample, each sensing element comprising:
a dock configured to attach to the first electrode, the dock comprising a dock nucleotide sequence, and an electrode bonding molecule;
an analyte capture complex, wherein a portion of the analyte capture complex is complementary to at least a portion of the dock nucleotide sequence, the analyte capture complex comprising a randomized aptamer sequence selected to interact with the target analyte, and one or more linker nucleotide sequences; and
an electroactive redox moiety attached to the analyte capture complex.

10. The sensing device of claim 9, wherein the analyte capture complex further comprises a first linker nucleotide sequence and a second linker nucleotide sequence, and wherein at least a portion of the first linker sequence is complementary to the second linker sequence.

11. The sensing device of claim 10, wherein the electroactive redox moiety is bound to one of the following: the first linker sequence, and the second linker sequence.

12. The sensing device of claim 9, wherein the second electrode further comprises a pair of electrodes configured to create an electric field across a cross-section of the channel.

13. The sensing device of claim 9, further comprising one of the following elements located at the second channel end: a selectively permeable membrane, or a channel cap.

14. (canceled)

15. The sensing device of claim 9, further comprising a reference sensor, comprising:
a plurality of reference aptamer sensing elements, each reference sensing element comprising:
a dock configured to attach to the electrode, the dock comprising a dock nucleotide sequence, and an electrode bonding molecule;
a reference analyte capture complex, wherein a portion of the reference analyte capture complex is complementary to at least a portion of the dock nucleotide sequence, the analyte capture complex comprising a deactivated randomized aptamer sequence, and one or more linker nucleotide sequences; and
an electroactive redox moiety; and
an electrode configured to detect redox events from the electroactive redox moiety

16. The device of claim 9, wherein the fluid sample is one of the following: sweat, interstitial fluid, blood, plasma, serum, tears, and saliva.

17. (canceled)
